(19) United States
(12) Patent Application Publication
(10) Pub. No.: US 2020/0079906 A1
(43) Pub. Date: Mar. 12, 2020

(54) PHOTODYNAMIC THERAPY COMPLEX

(71) Applicant: University College Cardiff Consultants Limited, Cardiff (GB)

(72) Inventors: Polina Prokopovich, Cardiff (GB); Stefano Perni, Cardiff (GB)

(21) Appl. No.: 15/772,882

(22) PCT Filed: Nov. 3, 2016
(86) PCT No.: PCT/GB2016/053407

§ 371 Date: May 2, 2018

(30) Foreign Application Priority Data
Nov. 3, 2015 (GB) 1519375.8

(51) Int. Cl. C08G 73/02; A61K 41/00; C08G 73/06

(52) U.S. Cl. CPC C08G 73/02; C08G 73/0633; C08G 73/0627; A61K 41/0057

(57) ABSTRACT

The invention concerns a complex for delivering light-activated antimicrobial agents (photosensitising (PS) agents) into cells; use of said complex as a medicament in photodynamic therapy; use of said complex for treatment of a microbial infection; a method for the manufacture of a composition comprising said complex; a pharmaceutical or veterinary composition comprising said complex; a combination therapeutic comprising said complex and at least one other agent; a method of treatment employing the use of said complex, composition or combination therapeutic. The complex comprises a poly-ß-amino ester and a photosensitizing agent.

### FIELD OF THE INVENTION

The invention relates to a complex for delivering light-activated antimicrobial agents (photosensitising agents) into cells; use of said complex as a medicament in photodynamic therapy; use of said complex for treatment of a microbial infection; a method for the manufacture of a composition comprising said complex; a pharmaceutical or veterinary composition comprising said complex; a combination therapeutic comprising said complex and at least one other agent; a method of treatment employing the use of said complex, composition or combination therapeutic.

### BACKGROUND OF THE INVENTION

Skin and soft-tissue infections (SSTIs) are the most frequent forms of methicillin-resistant Staphylococcus aureus (MRSA) infections and their rate has increased about 20 times in the last decade. Despite the media focus on MRSA other pathogen species have also developed resistance, i.e.

Vancomycin-Resistant Enterococci (VRE). SSTI incidence is 24.6 per 1000 person-years while among hospitalized patients the incidence is 7% to 10%. Non-antibiotic based antimicrobial therapies are therefore urgently needed in the management of infections in view of the rising number of microorganisms exhibiting resistance to one of more antibiotics.

Photodynamic therapy (PDT), sometimes called photochemotherapy, is a form of phototherapy using nontoxic light-sensitive compounds that are exposed selectively to light, whereupon they become toxic to targeted malignant and other diseased cells (phototoxicity). PDT has been proven to kill microbial cells including bacteria, fungi and viruses. It is a local, repeatable non-invasive technique which might be an effective alternative to antibiotics for the treatment of local infections.

Photodynamic therapy involves the use of a non-toxic dye, known as a photosensitizer (PS), which can be localized preferentially in certain cells or tissues and subsequently activated by the administration of light, of an appropriate wavelength, which leads to generation of reactive oxygen species (ROS). The mechanism of action of PDT derives from a light-induced excitation of the PS to a singlet state and its conversion to a triplet state, from the triplet state the energy is transferred either to oxygen generating ROS, such as singlet oxygen and free radicals, or to organic molecules; both these reactions results in the generation of radicals. In the context of PDT, two main pathways are characterized by different photochemical mechanisms labelled Type I (the excited PS reacts directly with the substrate) and Type II (the excited PS reacts directly with Oxygen). ROS are responsible for cytotoxic effects upon cells due to damage and destruction of cell walls, plasma membranes and DNA, hence leading to the death of the cells. PDT has found medical applications in cancer and infections antimicrobial-PDT (or aPDT) treatment.

Most modern PDT applications therefore involve three key components: a photosensitizer, a light source and reactive oxygen species. The combination of these three components leads to the chemical destruction of any tissues which have both selectively taken up the photosensitizer and have been locally exposed to light. In order to achieve the selective destruction of the target area using PDT, while leaving normal tissues untouched, either the photosensitizer can be applied locally to the target area, or photosensitive targets can be locally excited with light.

The application of aPDT to treat infection is based on the higher resistance of mammalian cells than pathogens to the oxidative stress induced by aPDT and by the biologically compatible PS. The use of light-activated antimicrobials has been demonstrated to be effective at killing a range of microorganisms such as bacteria (both Gram-positive and Gram-negative), fungi yeast and viruses. One of the key features of aPDT is its dual selectivity; (i) the antimicrobial effect is limited to the area that is treated afforded by PS localization, and (ii) light to bring about activation is anatomically confined, providing general low toxicity to the host and prevents disorder of the wider commensal microbial community. aPDT lethality is also not prone to induce resistance as bacteria are unlikely to be capable of simultaneously developing mechanisms to counteract all the possible damages caused.

A further benefit of aPDT is the possibility of inactivating not only viable cells but also virulence factors released by pathogens that can cause tissue damage after bacterial death compared to other antimicrobial techniques that are capable of only interacting with cells leaving virulence factors untouched.

Despite the numerous mentioned benefits of aPDT, the necessary exposure time (10-15 minutes) to effect antimicrobial action is too long for most current clinical applications and presents a substantial limitation. In order to improve the efficacy of aPDT, the encapsulation of PS into nanocarriers has been employed without great success and added concerns regarding the use of nanoparticles.

We disclose herein that the combination of photosensitizing agents with poly(β-amino esters (PBAEs), or derivatives thereof, shows vastly increased cellular uptake via acting as a carrier for said agents to improve delivery into the cell during PDT. Moreover, it has unexpectedly been found that combining the molecules in this way also leads to a significantly increased generation of free radicals leading to a markedly improved antimicrobial effect. This dual functionality thereby has the capacity to increase efficacy of photodynamic therapy and consequently reduce the exposure time and intensity required to achieve the targeted antimicrobial reduction. This has the potential to increase clinical effectiveness of the technique and represents a promising development in the area of antimicrobials.

### STATEMENTS OF INVENTION

According to a first aspect of the invention there is provided a complex comprising a poly-β-amino ester (PBAE), or a derivative thereof, and at least one photosensitizing agent.

PBAE is a polymer comprising poly-β-amino ester, or a derivative thereof.

PBAE refers to a polymer obtainable by reaction of a diacrylate monomer with an amine monomer. Suitable diacrylate monomers are compounds of formula (I)

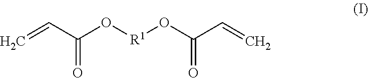

where R¹ is an optionally substituted divalent hydrocarbyl group, which may also be interposed with heteroatoms, with a primary or secondary amine monomer.

Suitable amine monomers include primary amines or secondary diamines such as those of formula (II) or (III)

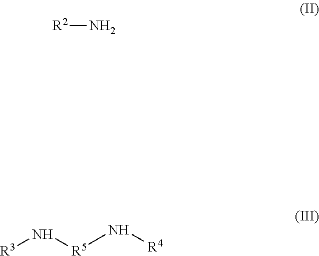

where R² is an optionally substituted hydrocarbyl group;

R³ and R⁴ are independently selected from optionally substituted hydrocarbyl groups;

R⁵ is a divalent hydrocarbyl group which is optionally substituted and may also be interposed with heteroatoms such as oxygen;

or R³ and/or R⁴ are linked together or to R⁵ to form one or more ring structures.

Suitable optional substituents for the hydrocarbyl groups R³, R⁴ and R⁵ comprise one or more groups selected from hydroxyl, C₁₋₆ alkoxy such as methoxy, C₁₋₆ alkylsilane or heterocyclic groups or halo.

Suitable optional substituents for R² are one or more groups selected from hydroxyl, C₁₋₆ alkoxy such as methoxy, C₁₋₆ alkylsilane or heteroaryl groups or halo.

Suitable, R² is an optionally substituted alkyl group, in particular an optionally substituted C₁₋₆ alkyl group wherein the optional substituents are as described above.

Suitably, R³ or R⁴ are optionally substituted alkyl groups, in particular unsubstituted C₁₋₆ alkyl such as methyl.

In a particular embodiment R³ and R⁴ are linked together to form a ring structure, in particular a 5-6-membered nitrogen containing ring such as a piperazine ring. In another particular embodiment, R³ and/or R⁴ are linked to R⁵ to form a piperidine ring.

Suitably R⁵ is an optionally substituted alkylene chain, in particular an unsubstituted C₁₋₁₀alkylene chain, such as ethylene.

Particular examples of monomers of formula (II) include 5-amino-1-pentanol, 2-methoxyethylamine, 3-(dimethylamino)-propylamine, (3-aminopropyl) triethoxysilane, 2-(2-pyridyl)ethylamine, 3-methoxy-propylamine, 3-amino-1,2-propanediol and 1-amino-2 propanol.

Particular examples of monomers of formula (III) include piperazine, 4,4-trimethylenedipiperidine and N,N-dimethylethyldiamine.

In a particular embodiment, the amine monomer is a compound of formula (III) or is a compound of formula (II) wherein R² carries at least one substituent which includes a nitrogen atom and/or is a hydroxyl group. Examples of such compounds are 4,4-trimethylenedipiperidine, 3-(dimethylamino)-propylamine 3-(dimethylamino)-propylamine, 2-(2-pyridyl)ethylamine and 1-amino-2 propanol.

Examples of PBAEs as described above may be represented by formula (IV) and (V)

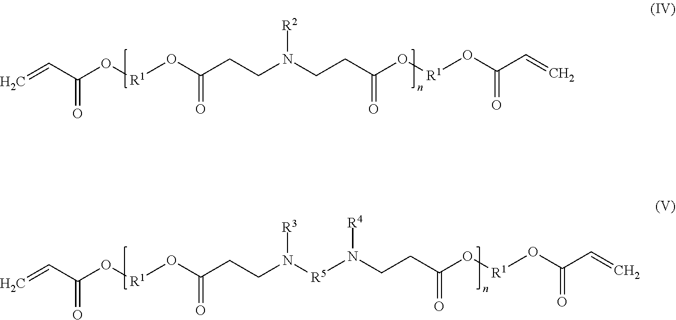

where R¹ is as defined above in relation to formula (I), R² is as defined above in relation to formula (II) and R³, R⁴ and R⁵ are as defined above in relation to formula (III); and n is a integer greater than 2, in particular in the range of from 10-100, for example from 30-70 such as from 45-55.

Suitable derivatives of the PBAEs have a functional group at an end of the polymer chains.

In a particular embodiment, the PBAE derivative carries amino functional groups at the ends of the polymer chains forming ‘end-caps’. Amino functional groups may comprise primary or secondary amino groups, able to link to an agent. Such functional groups or end caps may be introduced by reacting the PBAE with a primary or secondary diamine compound, for example a primary diamine of formula (VI)

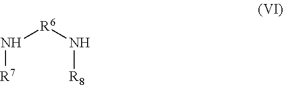

where R⁶ is an alkylene chain, and

R⁷ and R⁸ are the same and are selected from hydrogen or a C₁₋₆ alkyl group.

In a particular embodiment, R⁶ is a C₂₋₆ alkylene chain which is optionally interposed with a heteroatom, in particular a nitrogen atom.

In particular, R⁷ and R⁸ are both hydrogen.

Particular examples of compounds of formula (VI) are ethylene diamine and diethylenetriamine. Thus particular examples of R⁶ are ethylene and diethyleneamino. In a particular embodiment, the compound of formula (VI) is diethylenetriamine.

Thus suitable PBAE derivatives of the invention may be represented by formula (VII) or (VIII)

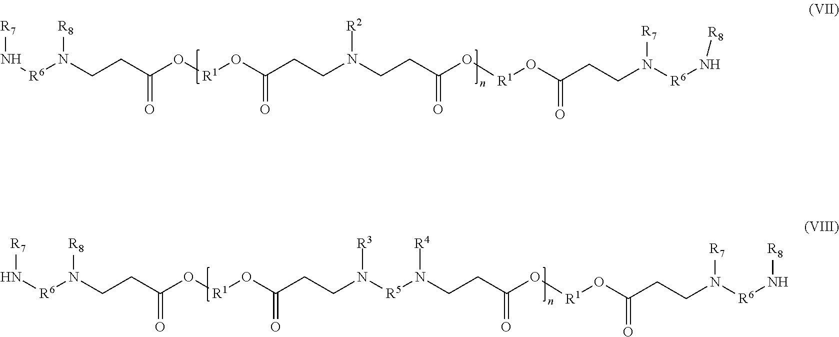

wherein R¹, R² R³, R⁴, R⁵, R⁶, R⁷, R⁸ and n are as defined above.

Particular groups R¹, R² R³, R⁴, R⁵, R⁶ and n are also as defined above.

Reference herein to a photosensitising agent refers to a light absorbing molecule that produces a chemical change in another adjacent molecule (often termed the acceptor or substrate) in a photochemical process. Preferably, said photosensitizer refers to a chemical compound that can be promoted to an excited state upon absorption light and undergoes intersystem crossing with oxygen to produce singlet oxygen. As will be apparent to those skilled in the art, generation of singlet oxygen species rapidly attacks any organic compounds it encounters, thus being highly cytotoxic.

Examples of photosensitising agents are well known to those skilled in the art and can broadly be divided into porphyrins, chlorophylls and dyes and include, but are not limited to, porphyrins, phthalocyanines, purpurins, chlorins, benzoporphyrins, lysomotropic weak bases, naphthalocyanines, cationic dyes, texaphyrins, pheophorbides, porphycenes, bacteriochlorins, ketochlorins, psoralens, xanthenes, thiazine compounds and derivatives and hematoporphyrin derivatives.

In a preferred embodiment of the invention, said photosensitising agent is a thiazine compound, porphyrins, chlorins, or xanthenes or derivatives thereof, including but not limited to (7-amino-8-methyl-phenothiazin-3-ylidene)-dimethyl-ammonium (TBO), Sn(IV)-chlorine-e6 (SnCe6), and O-Aminolevulinic acid (ALA), Rose Bengal (RB), Eosin Y (EOS) and Erythrosine B (ERI) or the like.

According to a further aspect of the invention there is provided a complex comprising a poly(β-amino ester (PBAE), or a derivative thereof, and at least one photosensitizing agent for use as a medicament in photodynamic therapy.

Photodynamic therapy is a term well known to those skilled in the art and refers is a two-step therapeutic process involving the administration of a photosensitizer (typically systematically or topically), followed by light illumination of an appropriate wavelength. For cytotoxic effects to take place, molecular oxygen must also be present. When these three factors are combined successfully (i.e. photosensitizer, light and oxygen), a photodynamic reaction occurs. The photodynamic reaction leads to generation of cytotoxic species, which cause cell death and tissue damage.

It has unexpectedly been discovered that the combination of photosensitisers and Poly-beta-amino esters (PBAEs), results in said PBAEs effecting improved cell delivery of said PS such that there is an increase of several orders of magnitude in photosensitiser uptake into cells. Further, it has been found that use of these complexes also leads to improved antimicrobial activity due to a heretofore undiscovered effect of same upon reactive oxygen species generation and therefore cell cytotoxicity, thereby improving the capacity for photochemical induced cell death. This dual functionality thereby has the capacity to increase the efficacy of photodynamic therapy and consequently reduce the exposure time and intensity required to achieve the targeted antimicrobial action.

According to a further aspect of the invention there is provided a complex comprising a poly-β-amino ester (PBAE), or a derivative thereof, and a photosensitizing agent, for use in the treatment of a microbial infection by photodynamic therapy.

It has been found in all organisms tested that when complexes as herein disclosed are brought into contact with a variety of microbes, said complex is rapidly internalised by the cell at an increased rate compared to photosensitiser alone. Further, following exposure to radiation, all polymer delivered photosensitizers showed improved cytotoxicity and antimicrobial effect. Notably, no effect upon the cell was observed in the absence of laser irradiation and thus demonstrates specificity. Therefore, complexes according to the invention have promising potential as antimicrobials in photodynamic therapy.

Reference herein to microbial infection refers to invasion and multiplication of pathogenic microbes in a subject including, but not limited to, a virus, bacterium, prion, fungus, viroid, or parasite that causes disease in its host. As will be appreciated by those skilled in the art, said infection may be symptomatic or asymptomatic and in the latter case, if left untreated, would result in advancing signs and/or symptoms indicative of the diseased or pathological state.

In a preferred embodiment of the invention, said antimicrobial infection refers to a bacterial infection including infection by both Gram positive and Gram negative bacteria.

As will be appreciated by those skilled in the art, reference herein to Gram-negative bacterial infection concerns infection with a bacterium that does not retain crystal violet dye in the Gram staining protocol. Examples of Gram-negative bacteria include, but are not limited to, the genera Acinetobacter, Aeromonas, Bacteroïdes, Bartonella, Bordetella, Brucella, Burkholderia, Campylobacter, Chlamydia, Chryseobacterium, Citrobacter, Enterobacter, Escherichia, Francisella, Fusobacterium, Haemophilus, Hafnia, Helicobacter, Klebsiella, Legionella, Moraxella, Morganella, Neisseria, Pantoea, Photobacterium, Proteus, Providencia, Pseudomonas, Salmonella, Serratia, Shigella, Stenotrophomonas, Veillonella, Vibrio, Yersinia.

As will be appreciated by those skilled in the art, reference herein to Gram-positive bacterial infection concerns an infection with a bacterium that does retain crystal violet dye in the Gram staining protocol. Examples of Gram-positive bacteria include, but are not limited to, the genera Actinomyces, Bacillus, Clostridium, Corynebacterium, Enterococcus, Lactobacillus, Lactococcus, Leuconostoc, Listeria, Micrococcus, Mycobacterium, Peptostreptococcus, Propionibacterium, Staphylococcus, Streptococcus, Streptomyces.

The invention also provides the use of a complex comprising a poly-β-amino ester (PBAE), or a derivative thereof, and at least one photosensitizing agent in the preparation of a composition for use in photodynamic therapy.

According to a further aspect of the invention there is provided a pharmaceutical or veterinary composition comprising the complex as defined herein, together with a pharmaceutically or veterinary acceptable excipient or carrier.

Suitable pharmaceutical excipients are well known to those of skill in the art. Pharmaceutical compositions may be formulated for administration by any suitable route, for example oral, rectal, nasal, bronchial (inhaled), topical (including eye drops, buccal and sublingual), vaginal or parenteral (including subcutaneous, intramuscular, intravenous and intradermal) administration and may be prepared by any methods well known in the art of pharmacy.

The composition may be prepared by bringing into association the complex as defined herein with the carrier. In general, the formulations are prepared by uniformly and intimately bringing into association the complex with liquid carriers or finely divided solid carriers or both, and then if necessary shaping the product. The invention extends to methods for preparing a pharmaceutical composition comprising bringing a complex comprising poly-β-amino ester (PBAE), or a derivative thereof, and at least one photosensitizing agent, as defined herein together in conjunction or association with a pharmaceutically or veterinarily acceptable carrier or vehicle.

Formulations for oral administration in the present invention may be presented as: discrete units such as capsules, sachets or tablets each containing a predetermined amount of the active agent; as a powder or granules; as a solution or a suspension of the active agent in an aqueous liquid or a non-aqueous liquid; or as an oil-in-water liquid emulsion or a water in oil liquid emulsion; or as a bolus etc.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate, stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

Parenteral formulations will generally be sterile.

For topical application to the skin, the composition may be made up into a cream, ointment, jelly, solution or suspension etc. Cream or ointment formulations that may be used for the drug are conventional formulations well known in the art, for example, as described in standard text books of pharmaceutics such as the British Pharmacopoeia.

In a preferred embodiment of this aspect of the invention the composition is formulated for topical application, ideally, in the form of a gel.

Typically, the dose of the complex or composition will be 10-1000 microM of PS. The precise amount of a composition as defined herein which is therapeutically effective, and the route by which such compound is best administered, is readily determined by one of ordinary skill in the art. Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The doses of the complex according to the invention administered to a subject can be chosen in accordance with different parameters, in particular in accordance with the mode of administration used and the state of the subject. As will be appreciated, the dose will also be determined by the nature and efficacy of the photosensitiser(s) that is/are used in the complex with the poly(β-amino ester (PBAE), or a derivative thereof. Other factors include the desired period of treatment. In the event that a response in a subject is insufficient at the initial doses applied, higher doses (or effectively higher doses by a different, more localized delivery route) may be employed to the extent that patient tolerance permits.

According to a yet further aspect of the invention there is provided a combination therapeutic comprising the above complex in combination with at least one other therapeutic. Ideally, said combination therapeutic is for use in the treatment of a microbial infection by photodynamic therapy.

We have found that the complex, as described herein, can be used to limit, treat or prevent infection whilst the host immune system clears same. Consequently, as will be appreciated by those skilled in the art, use of the complex in combination with other agents such as, but not limited to, amoebicides, antibiotics, antibody therapeutics, nanoparticles or other cytotoxic agents, will improve the relative efficacy of the fight against infection.

In a preferred embodiment of the invention said complex is used in combination with, either sequentially or simultaneously, at least one other therapeutic such as but not limited to amoebicides, antimicrobials including antibiotics, antibody therapeutics, nanoparticles or other cytotoxic agents.

According to a further aspect of the invention there is provided a method of treating a microbial infection by photodynamic therapy wherein a complex or composition or combination therapeutic according to the invention is administered to a subject having, or suspected of having a microbial infection.

As will be appreciated by those skilled in the art, treatment by photodynamic therapy involves delivering a complex, composition or combination therapeutic according to the invention to the cells to be treated whereby irradiation of the cells with light of a wavelength that can activate the complex, composition or combination therapeutic, i.e. the photosensitising agent therein, directly or indirectly generates reactive oxygen species which are cytotoxic to said cells. The light irradiation step to activate the photosensitising agent may take place according to techniques and procedures well known in the art. Further, the time for which the cells are exposed to said light in the methods of the present invention may vary according to the target, the photosensitizer (in the complex, composition or combination therapeutic of the invention), the amount of the photosensitizer accumulated in the target cells or tissue and the overlap/compatibility between the absorption spectrum of the photosensitizer and the emission spectrum of the light source. Appropriate light doses can be selected by a person skilled in the art.

In a preferred embodiment of this aspect of the invention, said subject is a mammal. Ideally said mammal is a primate. More ideally, said mammal is human, equine, canine, feline, porcine, ovine, ungulate or any other domestic or agricultural species. Yet most ideally, said mammal is human.

In yet a further preferred embodiment, said microbial infection is a viral, bacteral, prion, fungus, viroid, or parasitic infection. More preferably, said microbial infection is a bacterial infection including infection by Gram positive or Gram negative bacterium.

As will be appreciated by those skilled in the art, said method can be utilised in numerous disease context where there is suspected, or diagnosed, microbial infection, such as periodontal disease and dental infections including gum abscesses, periodontitis, gum disease, gingivitis and plaque biofilms, soft tissue and dermatological infections including wound infections, chronic wound ulcers, acne, burns, abcesses and mycosis, necrotising fasciitis, viral lesions including leishmaniasis and herpes keratitis, warts, and gangrene, fungal infections, cystitis, fungal infections, ear and eye infections.

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith. Moreover, unless stated otherwise, any feature disclosed herein may be replaced by an alternative feature serving the same or a similar purpose.

The invention will now be described by way of example only with reference to the Examples below and to the following Figures wherein:

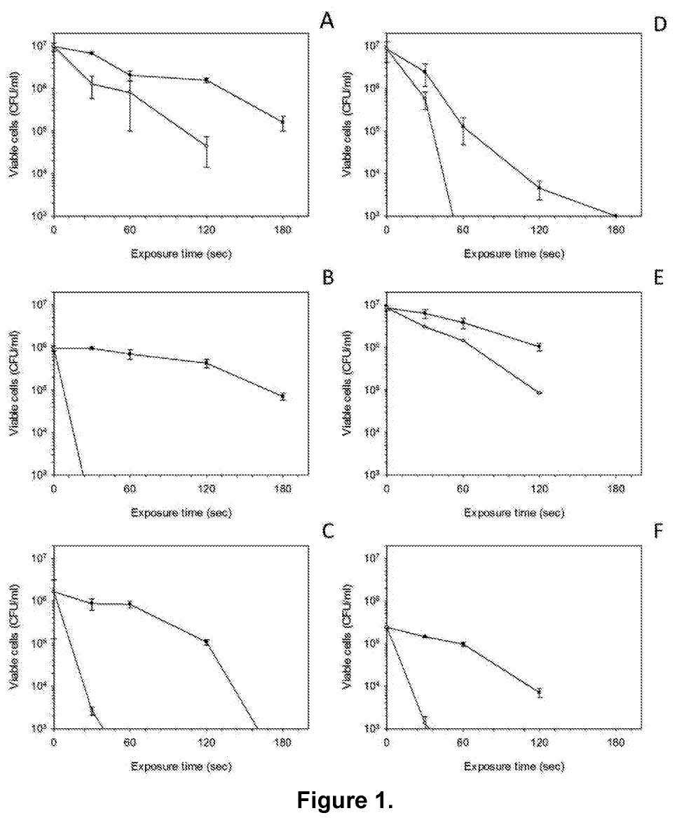

FIG. 1. Antimicrobial activity of TBO (●) and A3-e1-TBO (◯) against (a), E. coli (b), P. aeruginosa, (c) A. baumannii, (d) S. epidermidis, (e) MRSA and (f) S. pyogenes;

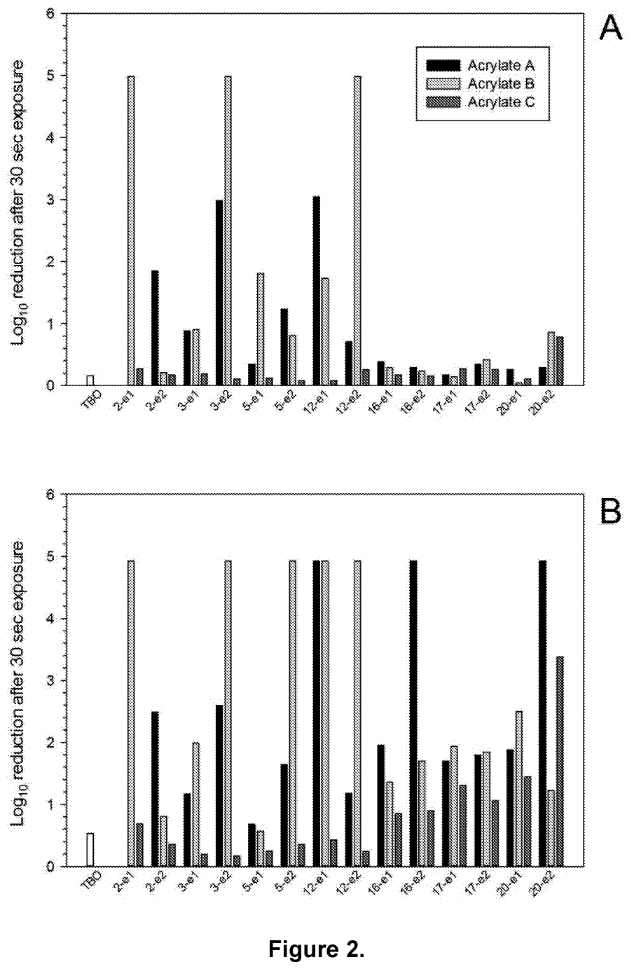

FIG. 2. Log₁₀ reduction of viable cells after 30 sec laser exposure of (a) E. coli and (b) S. epidermidis for different PBAE-TBO and pure TBO;

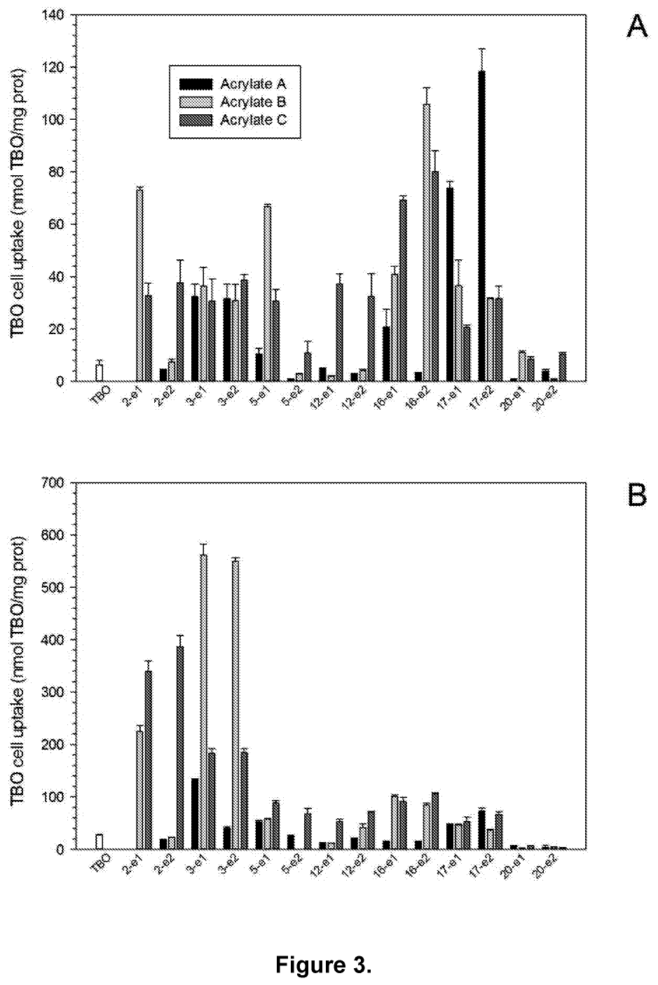

FIG. 3. Uptake of TBO for (a) E. coli and (b) S. epidermidis for different PBAE-TBO, having butandiole diacrylate (A), 1,6-hexanediol diacrylate (B) or tetra(ethylene glycol) diacrylate (C) shown in Table 2 below, compared to pure TBO;

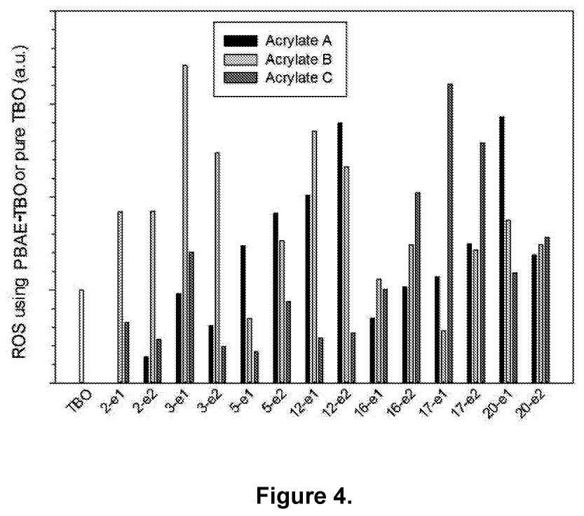

FIG. 4. ROS produced by different PBAE-TBO, having butandiole diacrylate (A), 1,6-hexanediol diacrylate (B) or tetra(ethylene glycol) diacrylate (C) shown in Table 2 below, after 1 minute irradiation compared to pure TBO measured in a solution of PBAE-TBO or pure TBO;

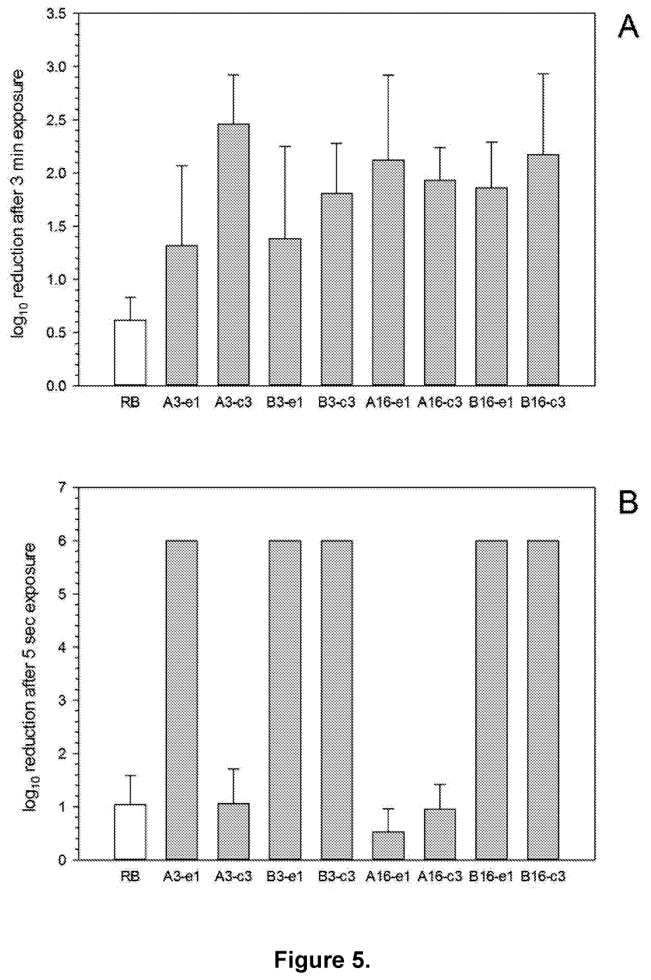

FIG. 5. Log₁₀ reduction of viable cells (a) E. coli after 3 min laser exposure and (b) MRSA NCTC12494 after 5 sec for different PBAE-Rose Bengal and pure Rose Bengal (RB);

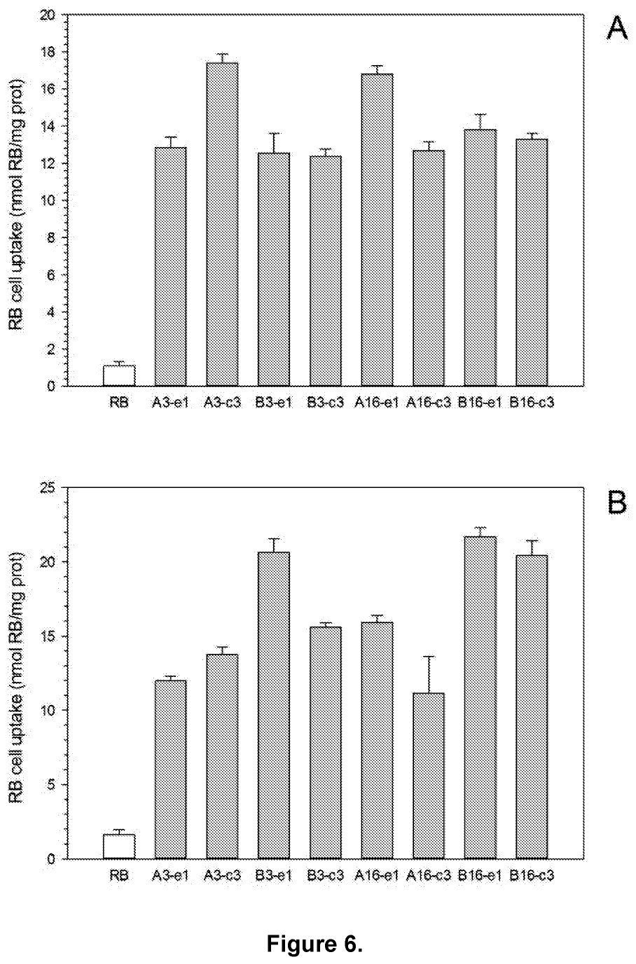

FIG. 6. Uptake of Rose Bengal (RB) for (a) E. coli and (b) MRSA NCTC12494 for different PBAE-RB and pure RB;

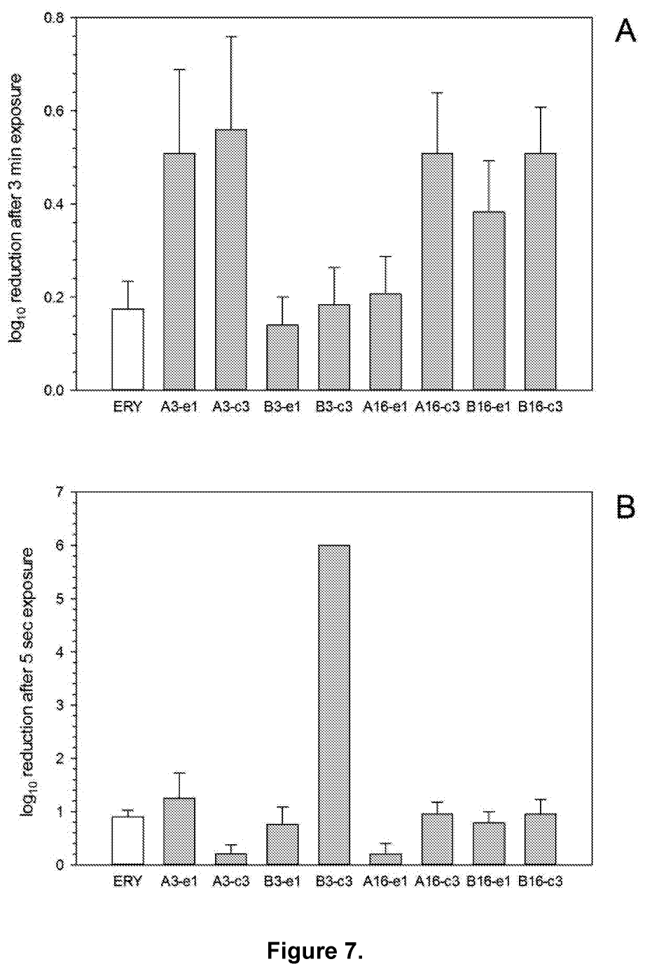

FIG. 7. Log₁₀ reduction of viable cells (a) E. coli after 3 min laser exposure and (b) MRSA NCTC12494 after 5 sec for different PBAE-Erythrosin B (ERY) and pure ERY;

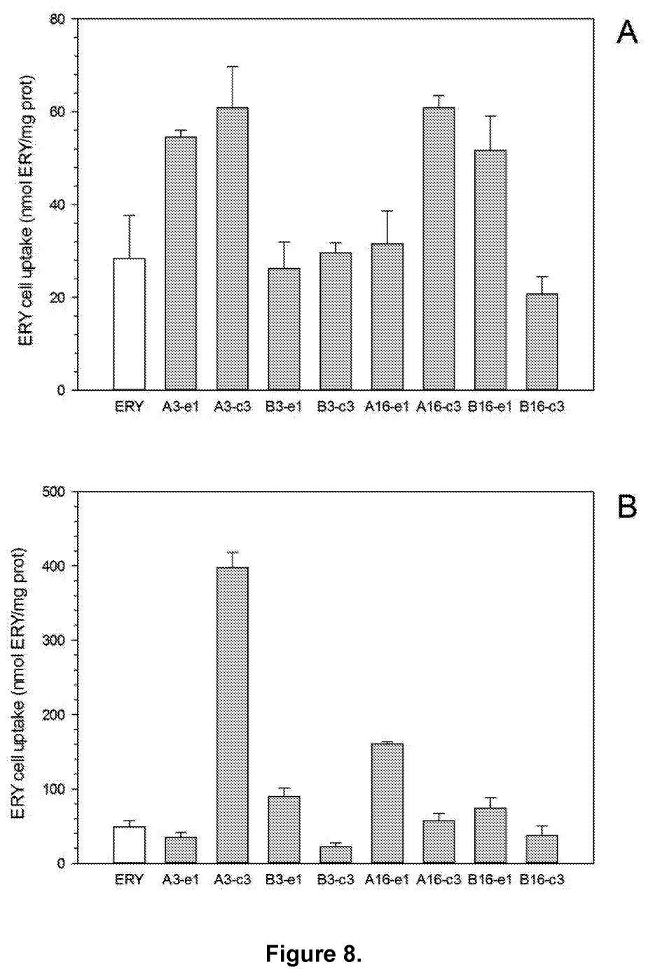

FIG. 8. Uptake of Erythrosin B (ERY) for (a) E. coli and (b) MRSA NCTC12494 for different PBAE-ERY and pure ERY;

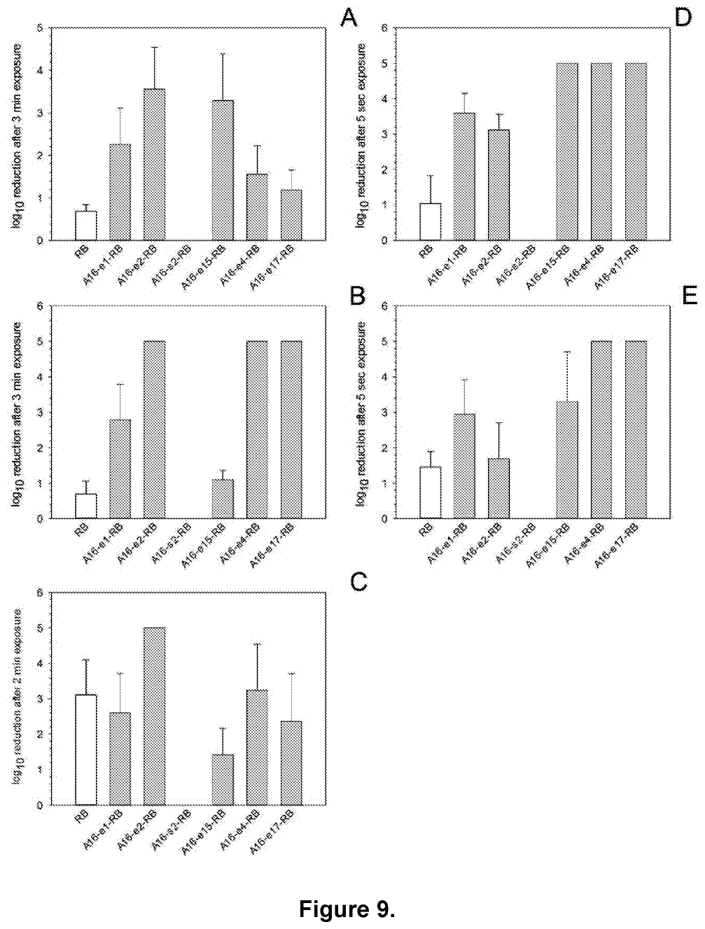

FIG. 9. Logic) reduction of viable cells (a) E. coli and (b) P. aeruginosa after 3 min laser exposure; (c) A. baumannii after 2 min laser exposure; (d) MRSA NCTC12494 and (e) MRSA 59275 after 5 sec for different PBAE-Rose Bengal and pure Rose Bengal;

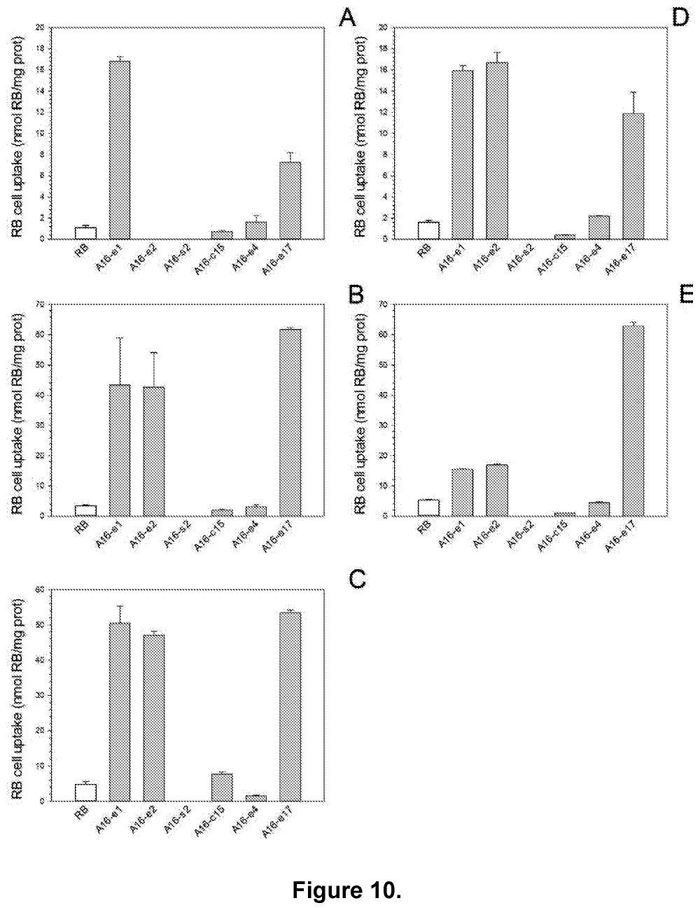

FIG. 10. Uptake of RB for cells (a) E. coli, (b) P. aeruginosa, (c) A. baumannii, (d) MRSA NCTC12494 and (e) MRSA 59275 for different PBAE-Rose Bengal (RB) and pure RB;

Table-1. List of amine monomers used to generate acrylate terminated poly(α-amino ester)s;

Table-2. Structure of 1,4-butandiole diacrylate (A), 1,6-hexanediol diacrylate (B) or tetra(ethylene glycol) diacrylate (C) used to generate poly(α-amino ester)s when mixed with the amine monomers of table 1;

Table-3. End-capping reagents used to generate PBAE derivatives;

Table-4. Structure of Toluidine Blue 0 (TBO), Rose Bengal (RB) and Erythrosin B (ERY).

Methods and Materials

Synthesis of PBAE

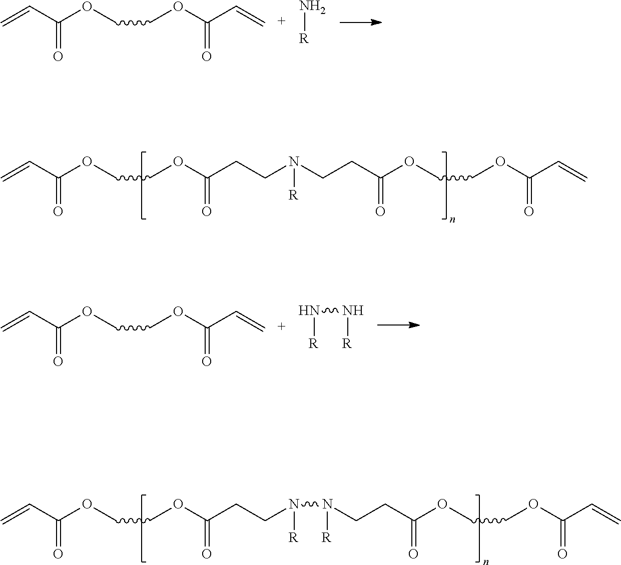

Acrylate-terminated poly(α-amino ester)s were synthesized by mixing 1,4-butandiole diacrylate (A), 1,6-hexanediol diacrylate (B) or tetra(ethylene glycol) diacrylate (C) shown in Table 2 below, with a range of amine monomers as shown in Table 1 below a 1.1:1 ratio diacrylate:amine in Dichloro-methane (DCM) at a concentration of 5 ml of DCM each 3.7 mmol of acrylate. In the above scheme, the letter ‘R’ is indicative of the particular organic group present in the monomers.

The polymerisation was then performed under stirring at 50° C. for 48 hours. PBAEs were precipitated through pouring the reaction mixture in about 10 times the volume of diethyl-ether under vigorous mixing; the solvent was removed under vacuum.

Preparation of End-Capped PBAE Derivatives

Acrylate-terminated polymers of were dissolved in DCM at a concentration of 31.13% w/w. A range of end capping agents shown in Table 3 below were then added.

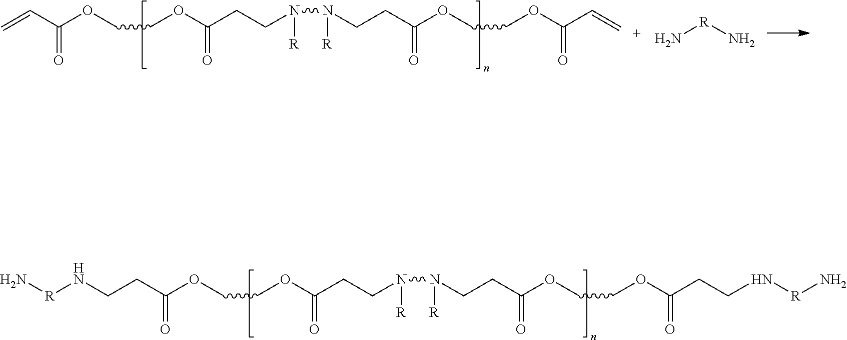

Specifically, amine capping reagents such as (either ethylene diamine (‘e1’), diethylenetriamine (‘e2’) were dissolved in DCM to a concentration of 0.25 mol/l). The capping reactions were performed by mixing the polymer/DCM solution with amine solution at a ratio of 800 μl per 321 mg of polymer solution; the mixture was kept for 24 hours at room temperature.

End-capped PBAEs were recovered through precipitation in diethyl-ether under vigorous mixing, the unreacted amine were removed centrifuging the suspension of PBAE in diethyl-ether/DCM for 2 min at 1155 g. The supernatant was removed and the PBAEs washed twice with diethyl-ether. The end-capped PBAEs were then dried under vacuum.

The PBAEs and derivatives obtained were coded through the constituents of the back bone using a capital letter to indicate the acrylate (A being 1,4-butanediol acrylate) and a number to indicate the amine as shown in Table 1;

the further end capping is described by a number preceded by the letter e. For example, A2-e1 is the PBAE obtained from 1,4-Butanediol diacrylate and 4,4′-Trimethylenedipiperidine then capped with ethylene-diamine.

The Preparation of Photosensitiser-Polymer Complex

10 mg of TBO, for example, photosensitiser (PS) was dissolved in DCM (25 ml) along with 100 mg of end-capped polymer; the solutions were immediately covered with aluminium foil and stirred for 24 hours at room temperature. The PBAEs-PS, e.g. PBAE-TBO, derivatives were recovered through precipitation in diethyl-ether under vigorous mixing, the unreacted molecules were removed centrifuging the suspension of PBAE in diethyl-ether/DCM for 2 min at 1155 g. The supernatant was removed and the PBAE-PS, e.g. PBAE-TBO, washed twice with a diethyl-ether/DCM mixture 4:1. The end-capped PBAEs were then dried under vacuum.

Antimicrobial Protocol

The organisms used in this study were Escherichia coli (NCTC10418) and Staphylococcus epidermidis (ATCC12228), Pseudomonas aeruginosa (NCIMB10548), Methicillin Resistant Staphylococcus aureus (NCTC12493), Streptococcus pyogenes (ATCC19615) and Acinetobacter baumannii (NCIMB9214); they were stored at −80° C. These organisms were subcultured, when needed, on Brain Heart Infusion (BHI) Agar overnight aerobically at 37° C. The plates were then stored at 4° C. for no more than two weeks. Bacterial suspensions used for the experiments were grown in Brain Heart Infusion (BHI) broth after inoculation with a loopful of cells from a single colony on a BHI plate and incubated aerobically for 24 h at 37° C. statically. These overnight cultures were then diluted 1 in 100 in PBS. The resulting bacterial suspension contained approximately 10⁶ CFU/mL. Each cell suspension was used to disperse pure PS, e.g. PBAE-TBO, to concentrations of 0.2 mg/mL or PBAEs-PS derivatives to an equivalent PS concentration.

200 μL aliquots of the resulting suspension were immediately poured in a GREINER 96 U-BOTTOM well plate. The well plate was then irradiated with a laser light (633 nm) using a 500 mW red laser (RRL-635 nm-500 mW-1080060, Changchun New Industries Optoelectronics, China) or a 500 mW green laser (MGL-FN-532 nm-500 mW-15097031, Changchun New Industries Optoelectronics, China) for different time periods (between 5 sec to 3 min). The red laser was employed for TBO while the green laser was used for RB and ERY.

After exposure the bacterial cells (L+S+) were counted through serial dilutions and then plating on BHI Agar. Along this test, different experiments were performed including the testing of dark toxicity (labelled with L−S+), samples exposed uniquely to laser light (L+S−) or samples not expose to either laser light and PBAE (L−S−).

Through the experiment prepared samples, well plates and inoculated plates were immediately covered with aluminium foil to reduce biased measuring results due to exposure of any light besides the laser. All plates were incubated after the experiment for 24 hours at 37° C. All experiments were performed on three independent cultures.

Reactive Singlet Oxygen Species

The generation of reactive oxygen species (ROS) was assessed using a singlet oxygen Sensor Green reagent (SOSG) (Molecular Probes, Unites States). The SOSG was stored at 20° C. and protected from light prior to use. The working solution was prepared with methanol to a final concentration of 5 mM. The prepared reagent was further diluted in methanol (1:100) before each experiment. Each sample was dispersed in PBS to a concentration equivalent to 0.2 mg/mL of pure PS, e.g. PBAE-TBO. 100 μL of diluted sample, 100 μL PBS and 20 μL reagent were immediately poured in two adjoining wells of a GREINER 96 U-BOTTOM well plate. The irradiation of one well (L+) was in each case done for a period of one minute with a 500 mW red laser (RRL-635nm-500nW-1080060, Changchun New Industries Optoelectronics, China); whilst the other well was covered with aluminium foil (L−). After exposure the ROS were determined using the FLUOstar OPTIMA (BMG Lab technologies, Germany).

Cell Uptake

10 mL of fresh sterile BHI broth were inoculated with a loopful of cells from a single colony on a BHI plate and incubated aerobically for 24 h at 37° C. statically. The bacterial suspension was then centrifuged with an Avanti J-20XP Centrifuge (Beckmann and Coulter, United States) for duration of 3 min at 2938 g, afterwards the supernatant was disposed. After one wash and centrifuge with PBS, 1 mL of sample containing either PBAE-PS or pure PS at an equivalent concentration of pure TBO or dye of 0.2 mg/mL was added to the precipitated cells. The resulting solution was vortexed and centrifuged after 3 min exposure. The exposure of samples to cells was limited to 3 min to ensure comparability to other experiments. After two more wash- and centrifugation-runs with PBS, the cells were dissolved in 1 mL of 0.1 M NaOH and 1% Sodium dodecyl sulfate (SDS) and then incubated at 37° C. for 24 h to lyase the cells.

The optical density of samples was measured at a 650 nm for TBO or 550 nm for RB and ERY using a plate reader (Labtech LT5000MS) against a calibration curve prepared using the corresponding bacterial lysate.

The protein content of the entire cell extract was determined by a modified Lowry method using bovine serum albumin (BSA) dissolved in 0.1 M NaOH/1% SDS to construct calibration curves. Results are expressed as nmol of PS/mg of cell protein.

Results

1.0 Antimicrobial Activity

We investigated the effect of tested samples in each bacterium upon bacterial growth. Furthermore, the comparison to diluted TBO is illustrated to evaluate effectiveness of each PS-polymer.

The CFU decreased gradually with the increase of irradiation time, which is emphasized by comparison to control samples. PBAE delivered photosensitiser was more potent in all bacterial samples tested compared to photosensitiser alone. Notably, no reduction in viable cells count was observed in samples exposed to PBAE-TBO without irradiation (L-S+).

1.1 A3-e1 Resulted in Significant Cell Death in Gram Positive and Gram Negative Bacteria

FIG. 1 displays the comparison of all tested microorganisms using pure TBO and A3-e1-TBO after exposure to laser light. In all cases, samples exposed to the polymer-TBO complex or to pure TBO without irradiation (L−) did not return any cell reduction over the time. In comparison, irradiated (L+) samples (L−) show tendencies of progressive reduction. The comparison with pure TBO showed an obvious improvement in the antimicrobial effect of laser exposure in all cases when the same amount of photosensitiser was employed along with A3-e1. Furthermore, when cells were exposed to laser light without the presence of photosensitiser (L+S−) did not reveal any antimicrobial activity. Generally, Gram+ were more readily inactivated than Gram-.

1.2 Alternative PBAEs Also Lead to Increased Photosensitisation

The comparison of the efficacy of other PBAE as delivery system of TBO for antimicrobial PDT against an example of Gram− (E. coli) and an example of Gram+(S. epidermidis) was tested in FIG. 2 using similar amine and end-capping agents for the preparation of PBAE. Generally, PBAE-TBO complex improved photosensitivity and cell death of the two bacteria tested with the acrylate B more effective than acrylate A; acrylate C largely returned the least improvement of the TBO activity.

2.0 PBAE Agents Improve Cell Uptake and Delivery of Photosensitisers

All PBAE tested increased the uptake of TBO in both E. coli and S. epidermidis (FIG. 3) cells suggesting that PBAE improves cell delivery. Acrylate B performed better than acrylate A in most of the cases and acrylate C the least effective. The overall efficiency of PBAE-TBO as delivery vehicle for TBO into bacterial cells depends on all three components of the polymer (acrylate, amine, and end-capping). The adjuvant effect of PBAE is remarkably greater in S. epidermidis than in E. coli, confirming the observed increased antimicrobial effect observed in Gram positive bacteria (FIG. 1).

3.0 Efficacy of PBAEs in Combination with Rose Bengal and Erythrosin

The red laser light (around 630 nm) is suitable to PS such as TBO, however other compounds require photons at different wavelength in order to act as PS.

For example, RB and ERY are excited by green laser light at around 530 nm. These PS were also found to exhibit antimicrobial activity against E. coli and MRSA, an example of Gram- and Gram+, respectively (FIGS. 5 and 7).

All PBAE tested in combination with RB were capable of improving the inactivation kinetic against both bacteria (FIG. 5), in many cases for MRSA the number of viable cells after irradiation fell below detection limit. No dark toxicity was detected for any of the PBAE.

ERY was not as effective as RB as light activated antimicrobial agent, however some of the PBAE tested did result in enhanced antimicrobial activity (FIG. 7).

Cell uptake of either RB or ERY (FIGS. 6 and 8) was increased when in combination with all the PBAE prepared, moreover no correlation was evident between cell uptake of the PS and inactivation rate.

4.0 Role of End-Capping Agent in Efficacy of PBAE-RB

The improvement in bacterial inactivation resulting from the combination of PS and PBAE depends not only on the nature of two monomers (acrylate and amine) but also on the end capping agent used. We investigated the effect of various chemicals suitable for PBAE end-capping on the efficacy of A16 (chosen based on the results in FIG. 5) combined with RB against a variety of Gram− and Gram+ bacteria also including a clinical isolate of MRSA; longer exposures were required for Gram− compared to Gram+ as the former are known to be more resistant to antimicrobial treatments.

FIG. 9 shows the log 10 reduction after exposure to green laser light for pure RB and for complexes A16-RB. The role of the end-capping agent on the modulation of the overall antimicrobial outcome is clearly noticeable. Some end-capping agents can enhance the antimicrobial activity of RBI; when the sulphur containing compound was used the product was not soluble hence no antimicrobial activity was displayed.

Cell uptake of RB (FIG. 10) was increased when in combination with some of the PBAE prepared, moreover no correlation was evident between cell uptake of the PS and inactivation rate.

5.0 Reactive Oxygen Species

The amount of ROS generated by pure TBO or by PBAE-TBO complexes is shown in FIG. 4 and reveals a strong dependence on the presence and structure of the PBAE. In most of the cases, acrylate B performed better than acrylate A.

No ROS were detected when only PBAE were exposed to laser light, hence PBAE are not photosensitisers. This demonstrates that PBAE delivery does not only act in enhancing TBO uptake by cells but also have a direct role in the mechanism of free radical formation by PDT.

### DISCUSSION

Light-activated antimicrobial agents (photosensitisers) are promising alternatives to antibiotics particularly, though not exclusively, for the treatment of skin infections and wounds through Photo Dynamic Therapy (PDT). Despite numerous benefits PDT applicability is limited by low efficacy requiring long light exposure time. We have developed a combination of photosensitisers with Poly-beta-amino esters (PBAEs) in order to enhance the photosensitiser uptake and ROS generating efficiency to reduce the exposure time required to achieve the targeted antimicrobial reduction. The overall performance of photosensitisers/PBAE complex is the result of two phenomena both of which PBAE is responsible for, one is cellular uptake and the other is the enhanced generation of ROS by the irradiated photosensitiser.

TABLE 1

| | | |
|---|---|---|
| 1 | 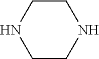 | piperazine |
| 2 | 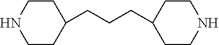 [SMILES: C(CC1CCNCC1)CC1CCNCC1] | 4,4′-Trimethylenedipiperidine |
| 3 | 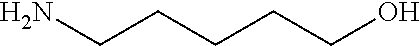 | 5-amino-1-pentanol |
| 4 | 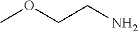 | 2-methoxyethylamine |
| 5 | 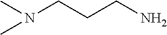 | 3-(dimethylamino)-1- propilamine |
| 6 | 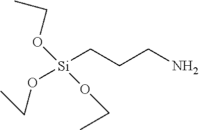 | (3-aminopropyl) triethoxysilane |
| 7 | 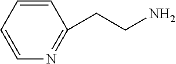 | 2-(2-pyridy)ethylamine |
| 8 | 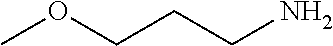 | 3-methoxy-propylamine |
| 9 | 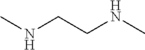 | N,N′-dimethylethyldiamine |
| 10 | 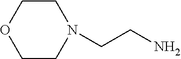 | 4-(2-aminoethyl)morpholine |
| 12 | 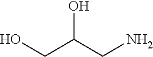 | 3-Amino-1,2-propanediol |
| 13 | 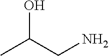 | DL 1-amino-2-propanol |
| 14 | 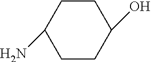 | trans-4-amino-hexanol |
| 15 | 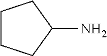 | Cyclopentylamine |
| 16 | 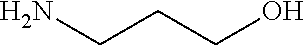 | 3-amino-1-propanol |
| 17 | 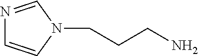 | 1-(3-Aminopropyl)imidazole |
| 18 | 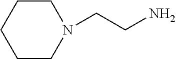 | 1-(2-aminoethyl)piperidine |
| 19 | 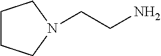 | 1-(2-aminoethyl)pyrrolidine |
| 20 | 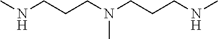 | N,N-bis[(3-methylamino) propyl]methylamine |

TABLE 2

| | | |
|---|---|---|
| A | 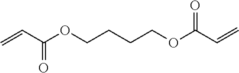 | 1,4-Butanediol diacrylate |
| B | 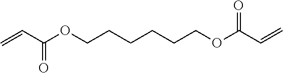 [SMILES: C=CC(=O)OCCCCCCOC(=O)C=C] | 1,6 Hexanediol diacrylate |
| C | 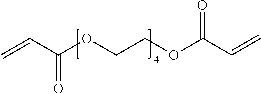 | Tetra(ethylene glycol) diacrylate |

TABLE 3

| | | |
|---|---|---|
| 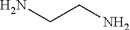 | Ethylen diamine | e1 |
| 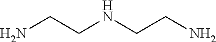 | Diethylentriamine | e2 |
| 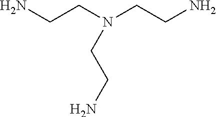 | Tris-(2-aminoethyl) amine | e3 |
| 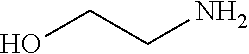 | 2-aminoethanol | c2 |
| 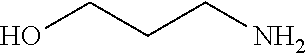 | 3-amino-1-propanol | c3 |
| 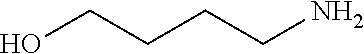 | 4-amino-1-butanol | c4 |
| 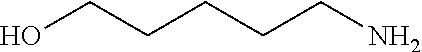 | 5-amino-1-pentanol | c5 |
| 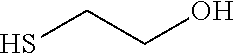 | 2-mercaptoethanol | s2 |
| 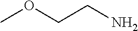 | 3-methoxy- ethyllamine | e4 |
| 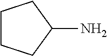 | Cyclopentylamine | e15 |
| 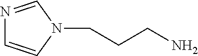 | 1-(3-Aminopropyl) imidazole | e17 |

TABLE 4

| | |
|---|---|
| 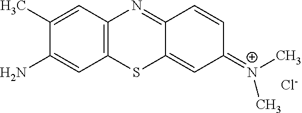 | Toluidine Blue O (TBO) |
| 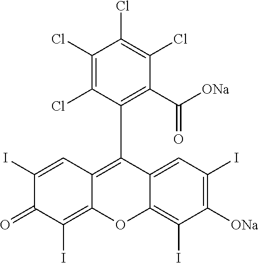 | Rose Bengal (RB) |
| 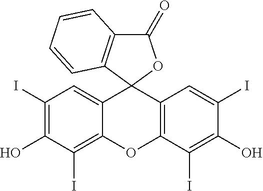 [SMILES: O=C1OC2(c3ccccc31)c1cc(I)c(O)c(I)c1Oc1c2cc(I)c(O)c1I] | Erythrosin B (ERY) |

1. A complex comprising a poly-β-amino ester (PBAE), or a derivative thereof, and at least one photosensitizing agent.

2. The complex according to claim 1 wherein said PBAE, or derivative thereof, is obtained by reaction of a diacrylate monomer with an amine monomer.

3. The complex according to claim 2 wherein said diacrylate monomer is a compound of formula (I) 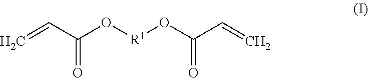
where R¹ is an optionally substituted divalent hydrocarbyl group, which may also be interposed with heteroatoms, with a primary or secondary amine monomer.

4. The complex according to claim 2 wherein said primary or secondary amine monomer is a compound of formula (II) or formula (III) 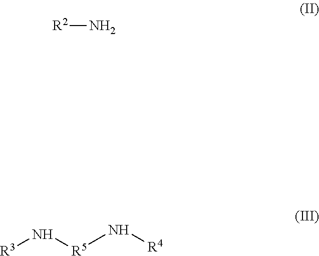
where R² is an optionally substituted hydrocarbyl group;
R³ and R⁴ are independently selected from optionally substituted hydrocarbyl groups;
R⁵ is a divalent hydrocarbyl group which is optionally substituted and may also be interposed with heteroatoms;
or R³ and/or R⁴ are linked together or to R⁵ to form one or more ring structures.

5. The complex according to claim 4 wherein optional substituents for the hydrocarbyl groups R³, R⁴ and R⁵ comprise one or more groups selected from hydroxyl, C₁₋₆alkoxy, C₁₋₆alkylsilane or heterocyclic groups or halo.

6. The complex according to claim 4 wherein optional substituents for R² are one or more groups selected from hydroxyl, C₁₋₆alkoxy, C₁₋₆alkylsilane or heteroaryl groups or halo.

7. The complex according to claim 4 wherein R² is an optionally substituted alkyl group, in particular an optionally substituted C₁₋₆alkyl group.

8. The complex according to claim 4 wherein R³ or R⁴ are substituted alkyl groups.

9. The complex of claim 8 wherein R³ or R⁴ are optionally substituted alkyl groups, in particular unsubstituted C₁₋₆alkyl.

10. The complex according to claim 4 wherein R³ and R⁴ are linked together to form a ring structure.

11. The complex according to claim 10 wherein said ring structure is a piperazine or piperidine ring.

12. The complex according to claim 4 wherein said amine monomer is selected from 5-amino-1-pentanol, 2-methoxyethylamine, 3-(dimethylamino)-propylamine, (3-aminopropyl) triethoxysilane, 2-(2-pyridyl)ethylamine, 3-methoxy-propylamine, 3-amino-1,2-propanediol, 1-amino-2 propanol, piperazine, 4,4-trimethylenedipiperidine and N,N-dimethylethyldiamine.

13. The complex according to claim 12 wherein the amine monomer is a compound of formula (III) or is a compound of formula (II) wherein R² carries at least one substituent which includes a nitrogen atom and/or is a hydroxyl group.

14. The complex according to claim 13 wherein the amine monomer is selected from the group comprising: 4,4-trimethylenedipiperidine, 3-(dimethylamino)-propylamine 3-(dimethylamino)-propylamine, 2-(2-pyridyl)ethylamine and 1-amino-2 propanol.

15. The complex according to claim 1 wherein said PBAE is represented by formula (IV) and (V) 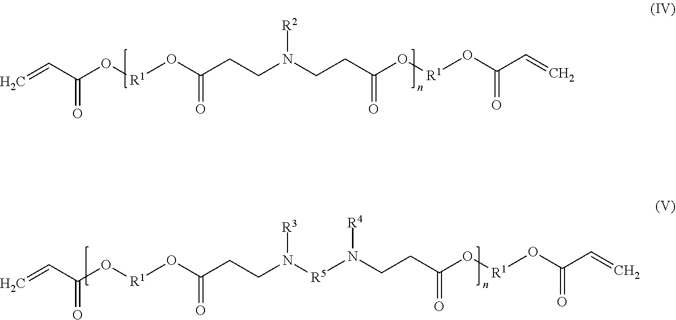
where R¹ is an optionally substituted divalent hydrocarbyl group, which may also be interposed with heteroatoms, with a primary or secondary amine monomer, R² is an optionally substituted hydrocarbyl group; and R³ and R⁴ are independently selected from optionally substituted hydrocarbyl groups; R⁵ is a divalent hydrocarbyl group which is optionally substituted and may also be interposed with heteroatoms;
or R³ and/or R⁴ are linked together or to R⁵ to form one or more ring structures; and n is a integer greater than 2.

16. The complex according to claim 15 wherein n is in the range of from 10-100.

17. The complex according to claim 16 wherein n is in the range of 30-70 or 45-55.

18. The complex according to claim 1 wherein said PBAE is a derivative having a functional group at an end of the polymer chains.

19. The complex according to claim 18 wherein said functional groups are introduced by reacting the PBAE with a primary or secondary diamine compound.

20. The complex according to claim 19 wherein said primary or secondary diamine compound is of formula (VI) 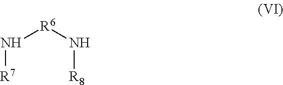
where R⁶ is an alkylene chain, and
R⁷ and R⁸ are the same and are selected from hydrogen or a C₁₋₆alkyl group.

21. The complex according to claim 20 wherein said primary or secondary compound is selected from the group consisting of: ethylene, diethyleneamine, and diethylenetriamine.

22. The complex according to claim 18 wherein said PBAE derivatives of the invention may be represented by formula (VII) or (VIII) 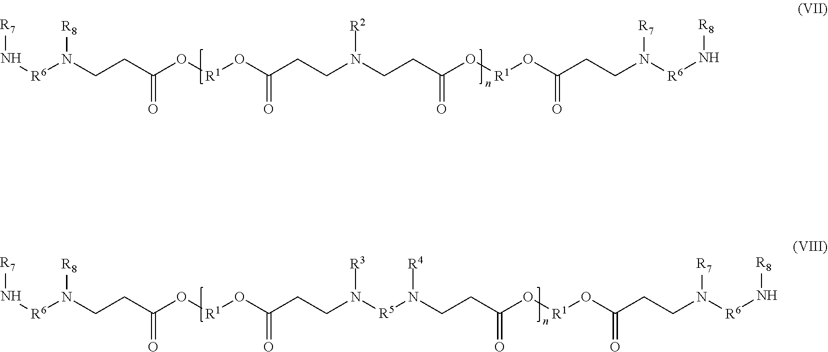
wherein R¹ is an optionally substituted divalent hydrocarbyl group, which may also be interposed with heteroatoms, with a primary or secondary amine monomer;
R² is an optionally substituted hydrocarbyl group;
R3 and R4 are independently selected from optionally substituted hydrocarbyl groups;
R⁵, R5 is a divalent hydrocarbyl group which is optionally substituted and may also be interposed with heteroatoms;
or R3 and/or R4 are linked together or to R5 to form one or more ring structures.
R⁶, R6 is an alkylene chain; and
R7 and R8 are the same and are selected from hydrogen or a C1-6alkyl group.

23. The complex according to claim 1 wherein said photosensitizing agent is selected from the group consisting of: thiazine compound, porphyrins, chlorins, and xanthenes or derivatives thereof.

24. The complex according to claim 23 wherein said photosensitizing agent is selected from the group consisting of: (7-amino-8-methyl-phenothiazin-3-ylidene)-dimethyl-ammonium (TBO), Sn(IV)-chlorine-e6 (SnCe6), and δ-Aminolevulinic acid (ALA), Rose Bengal (RB), Eosin Y (EOS) and Erythrosine B (ERI).

25-28. (canceled)

29. A pharmaceutical or veterinary composition comprising a complex according to claim 1 together with a pharmaceutically or veterinary acceptable excipient or carrier.

30. The pharmaceutical or veterinary composition according to claim 29 wherein said composition is formulated for topical application.

31. A combination therapeutic comprising the complex according to claim 1 in combination with at least one other therapeutic.

32. (canceled)

33. A method of treating a microbial infection by photodynamic therapy comprising administering the complex according to claim 1, a pharmaceutical or veterinary composition comprising the complex and a pharmaceutically or veterinary acceptable excipient or carrier, or a combination therapeutic comprising the complex and at least one other therapeutic, to a subject having or suspected of having a microbial infection.

34. The method according to claim 33 wherein said subject is a mammal selected from the group comprising: human, equine, canine, feline, porcine, ovine, ungulate or any other domestic or agricultural species.

35. The method according to claim 34 wherein said subject is human.

36. The method according to claim 33 wherein said microbial infection is a viral, bacterial, prion, fungus, viroid, or parasitic infection.

37. The method according to claim 36 wherein said microbial infection is a bacterial infection.
